(19) United States
(12) Patent Application Publication
(10) Pub. No.: US 2009/0326000 A1
(43) Pub. Date: Dec. 31, 2009

(54) RESVERATROL FORMULATIONS AND METHODS OF USE

(76) Inventors: Christopher Hull, Van Nuys, CA (US); Jeffrey Feliciano, Paso Robles, CA (US)

Correspondence Address: ITALIA IP, 3500 WEST OLIVE AVE., SUITE 300, BURBANK, CA, 91505, US

(21) Appl. No.: 12/147,851

(22) Filed: Jun. 27, 2008

(51) Int. Cl. A61K 31/437; A61K 31/047; A61P 3/04; A61P 29/00

(52) U.S. Cl. 514/299; 514/729

(57) ABSTRACT

A method is provided for preventing or treating obesity and for reducing the percentage body fat content of an individual. The method involves administration, preferably topical administration, of an active agent selected from the group consisting of resveratrol, pharmacologically acceptable salts, esters, amides, and analogs thereof, and combinations of any of the foregoing. Topical formulations for use in conjunction with the method for reducing the percentage body fat content of an individual are provided as well.

### TECHNICAL FIELD

The present invention relates generally to topical formulations, their methods of use and manufacture thereof. More particularly, the invention relates to formulations of resveratrol that are useful, for example, in the prevention and treatment of obesity and in reducing the percentage body fat content of an individual.

### BACKGROUND

An individual's percentage body fat content is the total weight of the individual's fat divided by the individual's weight and reflects both essential fat and storage fat. Essential fat is defined as the amount necessary for maintenance of life and reproductive functions. Therefore the percentage body fat content for women is greater than that for men, due to the demands of childbearing and other hormonal functions. In men, the essential fat range is 2-5%, and it's 10-13% in women.

The American Council on Exercise has categorized ranges of body fat percentages as follows:

| Description | Women | Men |
|---|---|---|
| Essential fat | 12-15% | 2-5% |
| Athletes | 16-20% | 6-13% |
| Fitness | 21-24% | 14-17% |
| Acceptable | 25-31% | 18-25% |
| Obese | 32%+ | 25%+ |

The essential fat values in the table above are considerably lower than the recommended minimum body fat percentage levels. A certain amount of stored fat is required to be available as fuel for the body in times of stress and need. It is inconclusive as to whether falling in a particular category of these body fat percentages is better or worse for an individual's health than any other. But the higher an individual's measured body fat percentage above average levels, the higher the health risk for weight related illness, like heart disease, high blood pressure, gallstones, type 2 diabetes, osteoarthritis, and certain cancers. Also, the higher an individual's percentage of body fat, the fewer the calories needed to maintain the current weight and, as a result, it is much easier to gain weight. The reason fewer calories are needed is simply because muscle requires more calories because of its density versus less dense fat tissue.

It has been estimated that extra weight costs the United States as much as $93 billion in annual medical bills, with the government paying almost half of that amount. This is a record high estimate of the medical costs of overweight and obesity. It is larger than the annual medical costs associated with cigarette smoking, which was estimated at about $76 billion a few years ago.

Almost two-thirds of the people in the USA are either overweight or obese. The definition of overweight is roughly 10 to 30 pounds over a healthy weight and obesity is defined as 30 or more pounds over.

The difficulties of losing weight and maintaining that weight loss have been well documented in the mainstream media. This phenomenon is so prevalent it has been given names such as “yo-yo” dieting or “boomerang fat”. Individual's can spend countless amounts of time and effort to control their weight, with little or no visible results.

It has now been discovered that the topical application of a formulation comprising resveratrol, its pharmacologically acceptable salts, esters, amides, and analogs thereof, can improve the rate and amount of body fat lost by an individual.

Resveratrol is a phytoalexin and belongs to a class of polyphenolic compounds called stilbenes. Resveratrol is produced naturally by several plants in response to stress, injury, fungal infection, and ultraviolet (UV) radiation. Resveratrol is a fat-soluble compound that occurs in a trans and a cis configuration which have also been produced by chemical synthesis. Scientists became interested in resveratrol after a number of beneficial health effects, such as anti-cancer, antiviral, neuroprotective, anti-aging, and anti-inflammatory effects have been reported, but all of these studies are “in-vitro” (test tube) or in yeast, worms, fruit flies, fish, mice, and rats.

Resveratrol is found in the skin of red grapes and is a constituent of red wine but, based on extrapolation from animal trials, apparently not in sufficient amounts to explain the “French paradox” that the incidence of coronary heart disease is relatively low in southern France despite high dietary intake of saturated fats.

To date, topical administration of resveratrol has been shown to be effective in preventing or treating a certain limited number of skin conditions, disorders and diseases, e.g., skin conditions associated with inflammation, skin damage associated with exposure to the sun, and the effects of natural aging.

### SUMMARY

One embodiment of the invention provided herein is a composition for reducing the percentage body fat content of an individual comprising at least one agent selected from the group consisting of resveratrol, its pharmacologically acceptable salts, esters, amides, analogs and mixtures thereof, and at least one trans-stilbene, its pharmacologically acceptable salts, esters, amides, analogs and mixtures thereof.

Also provided herein is a method for reducing the percentage body fat content of an individual comprising application of said composition to the skin of the individual, and a method for producing the composition for reducing the percentage body fat content of an individual.

Further provided herein, is a composition for reducing inflammation of joints, muscles and skin comprising at least one agent selected from the group consisting of resveratrol, its pharmacologically acceptable salts, esters, amides, and analogs thereof, and at least one trans-stilbene, its pharmacologically acceptable salts, esters, amides, analogs and mixtures thereof.

### DETAILED DESCRIPTION

One embodiment of the present invention is directed to compositions for reducing the percentage body fat content of an individual which has been found to comprise, a) at least one agent selected from the group consisting of resveratrol, its pharmacologically acceptable salts, esters, amides, analogs and mixtures thereof; and b) at least one trans-stilbene, its pharmacologically acceptable salts, esters, amides, analogs and mixtures thereof.

A more preferable embodiment of the present invention is directed to formulation comprising a) at least one agent selected from the group consisting of resveratrol, its pharmacologically acceptable salts, esters, amides, analogs and mixtures thereof; b) at least one trans-stilbene, its pharmacologically acceptable salts, esters, amides, analogs and mixtures thereof; c) at least one forskohlin fat burner; d) at least one alkyl alcohol; e) at least one aryl alcohol; f) at least one ketone; and g) at least one emulsifier.

A particularly preferable embodiment of the present invention is directed to formulation comprising a) at least one agent selected from the group consisting of resveratrol, its pharmacologically acceptable salts, esters, amides, analogs and mixtures thereof; b) at least one trans-stilbene, its pharmacologically acceptable salts, esters, amides, analogs and mixtures thereof; c) at least one forskohlin fat burner; d) at least one alkyl alcohol; e) at least one aryl alcohol; f) at least one ketone; g) at least one emulsifier; and h) at least one stimulant based on a plant extract.

A variety of additives can be incorporated into the inventive compositions for their intended functions. These additives are usually used in small amounts.

Useful additives include but are not limited to, for example, emulsifiers, fragrances, oils, caffeine, yohimbe, sunscreens, and the sodium salt of pyrrolidone carboxylic acid.

### EXAMPLE 1

### Method of Formulation

Into a 1 liter vessel equipped with a method for heating and stirring, added 560 grams of ethanol. Heated the vessel and ethanol to 80° F. to 100° F. Added 3 grams of benzyl alcohol to the 1 liter vessel. Added 8 grams of at keto-isoLeucine to the 1 liter vessel. Added 3 grams of Eucalyptus Oil to the 1 liter vessel. Added 12 grams of resveratrol to the 1 liter vessel. Added 6 grams of trans-stilbene enhancer to the 1 liter vessel. Added 5 grams of tetrahydrocurcuminoid to the 1 liter vessel. Cooled the vessel and contents to room temperature. Filtered the contents of the vessel through a #5 and a #2 filter paper to obtain the topical pre-spray formulation for reducing the percentage body fat content of an individual.

### EXAMPLE 2

### Method of Formulation

Into a 1 liter vessel equipped with a method for heating and stirring, added 560 grams of ethanol. Heated the vessel and ethanol to 80° F. to 100° F. Added 3 grams of benzyl alcohol to the 1 liter vessel. Added 8 grams of at keto-isoLeucine to the 1 liter vessel. Added 3 grams of Eucalyptus Oil to the 1 liter vessel. Added 12 grams of resveratrol to the 1 liter vessel. Added 6 grams of trans-stilbene enhancer to the 1 liter vessel. Added 3 grams of forskohlin fat burner to the 1 liter vessel. Added 3 grams of caffeine to the 1 liter vessel. Added 1 gram yohimbe to the 1 liter vessel. Cooled the vessel and contents to room temperature. Filtered the contents of the vessel through a #5 and a #2 filter paper to obtain the topical fat burning formulation for reducing the percentage body fat content of an individual.

### EXAMPLE 3

### Application of Formulation

Each morning a 36 year old male who is 6 feet 1 inch in height and weighed 212 pounds applied approximately 0.30 ounces of the topical pre-spray formulation produced in Example 1, via a spray bottle to his whole body. Each evening the 36 year old male applied 0.30 ounces of a solution of the topical fat burning formulation produced in Example 2, via spray bottle to localized problem areas of his body, i.e., the abdomen. At the end of six months the 36 year old male had reduced his percentage body fat content from 25% to 12%, while increasing his weight to 225 pounds. Surprisingly, the 36 year old male further observed a reduction in the soreness and inflammation of his muscles, joints, and skin with the application of the formulations of Examples 1 and 2.

What is claimed is:

1. A composition for reducing the percentage of body fat content of an individual comprising:
a) at least one agent selected from the group consisting of resveratrol, its pharmacologically acceptable salts, esters, amides, and analogs thereof; and
b) at least one trans-stilbene, its pharmacologically acceptable salts, esters, amides, analogs and mixtures thereof.

2. A composition according to claim 1, wherein the at least one agent selected from the group consisting of resveratrol, its pharmacologically acceptable salts, esters, amides, and analogs thereof; is present in the range of about 1.0% to about 3.0% weight based on the total weight of the composition.

3. A composition according to claim 1, wherein the at least one trans-stilbene its pharmacologically acceptable salts, esters, amides, analogs and mixtures thereof, is present in the range of about 0.50% to about 2.0% weight based on the total weight of the composition.

4. A composition for reducing the percentage of body fat content of an individual comprising:
a) at least one agent selected from the group consisting of resveratrol, its pharmacologically acceptable salts, esters, amides, analogs and mixtures thereof;
b) at least one trans-stilbene, its pharmacologically acceptable salts, esters, amides, analogs and mixtures thereof;
c) at least one forskohlin fat burner;
d) at least one alkyl alcohol;
e) at least one aryl alcohol;
f) at least one ketone; and
g) at least one emulsifier.

5. A composition according to claim 4, wherein the at least one alkyl alcohol is present in the range of about 85% to about 95% weight based on the total weight of the composition.

6. A composition according to claim 4, wherein the at least one forskohlin fat burner is present in the range of about 0.10% to about 1.0% weight based on the total weight of the composition.

7. A composition according to claim 4, wherein the at least one aryl alcohol is present in the range of about 0.10% to about 1.0% weight based on the total weight of the composition.

8. A composition according to claim 4, wherein the at least one emulsifier is present in the range of about 0.10% to about 1.0% weight based on the total weight of the composition.

9. A composition according to claim 4, further comprising caffeine in the range of about 0.10% to about 1.0% weight based on the total weight of the composition.

10. A method for reducing the percentage body fat content of an individual comprising the steps of:
i) applying to the individual's skin a therapeutically effective amount of a topical pre-spray formulation, wherein the topical pre-spray formulation comprises:
a) at least one agent selected from the group consisting of resveratrol, its pharmacologically acceptable salts, esters, amides, analogs and mixtures thereof; and
b) at least one trans-stilbene, its pharmacologically acceptable salts, esters, amides, analogs and mixtures thereof; and
ii) applying to the individual's skin a therapeutically effective amount of a topical fat burning formulation, wherein the topical fat burning formulation comprises
a) at least one agent selected from the group consisting of resveratrol, its pharmacologically acceptable salts, esters, amides, analogs and mixtures thereof;
b) at least one trans-stilbene, its pharmacologically acceptable salts, esters, amides, analogs and mixtures thereof;
c) at least one forskohlin fat burner;
d) at least one alkyl alcohol;
e) at least one aryl alcohol;
f) at least one ketone; and
g) at least one emulsifier.

11. A method for producing a composition for reducing the percentage body fat content of an individual, comprising the steps of, providing a vessel, adding at least one alkyl alcohol to the vessel, heating the vessel and at least one alkyl alcohol to 80° F. to 100° F., adding at least one aryl alcohol to the vessel, adding at least one ketone to the vessel, adding at least one emulsifier to the vessel, adding at least one agent selected from the group consisting of resveratrol, its pharmacologically acceptable salts, esters, amides, analogs and mixtures thereof, to the vessel, adding at least one trans-stilbene its pharmacologically acceptable salts, esters, amides, analogs and mixtures thereof, to the vessel, adding at least one forskohlin fat burner, cooling the vessel and contents to room temperature, filtering the contents of the vessel through a #5 and a #2 filter paper to obtain the composition for reducing the percentage body fat content of an individual.

12. A method according to claim 11, wherein the at least one alkyl alcohol is added in an amount in the range of about 85% to about 95% weight based on the total weight of the composition.

13. A method according to claim 11, wherein the at least one agent selected from the group consisting of resveratrol, its pharmacologically acceptable salts, esters, amides, analogs and mixtures thereof; is added in an amount in the range of about 1.0% to about 3.0% weight based on the total weight of the composition.

14. A method according to claim 11, wherein the at least one aryl alcohol is added in an amount in the range of about 0.10% to about 1.0% weight based on the total weight of the composition.

15. A method according to claim 11, wherein the at least one ketone is added in an amount in the range of about 0.10% to about 1.0% weight based on the total weight of the composition.

16. A method according to claim 11, wherein the at least one emulsifier is added in an amount in the range of about 0.10% to about 1.0% weight based on the total weight of the composition.

17. A method according to claim 11, wherein the at least one trans-stilbene, its pharmacologically acceptable salts, esters, amides, analogs and mixtures thereof, is added in an amount in the range of about 0.50% to about 2.0% weight based on the total weight of the composition.

18. A method according to claim 11, wherein the at least one forskohlin fat burner is added in an amount in the range of about 0.10% to about 1.0% weight based on the total weight of the composition.

19. A composition for reducing inflammation:
a) at least one agent selected from the group consisting of resveratrol, its pharmacologically acceptable salts, esters, amides, and analogs thereof; and
b) at least one trans-stilbene, its pharmacologically acceptable salts, esters, amides, analogs and mixtures thereof.

20. A method for reducing inflammation comprising the step of applying a composition according to claim 19 to the individual's skin over the inflamed joints and muscles.
